(19) United States
(12) Patent Application Publication
(10) Pub. No.: US 2018/0296522 A1
(43) Pub. Date: Oct. 18, 2018

(54) ENHANCED EXPRESSION OF mTOR AND INHIBITED EXPRESSION OF MYOSTATIN IN SKELETAL MUSCLE CELLS

(71) Applicant: IN Ingredients, Inc., Columbia, TN (US)

(72) Inventors: Bolin Qin, Gaithersburg, MD (US); Augustin T. Romero, Spring Hill, TN (US)

(73) Assignee: IN Ingredients, Inc., Columbia, TN (US)

(21) Appl. No.: 15/524,742

(22) PCT Filed: Nov. 6, 2015
(86) PCT No.: PCT/US2015/059452

§ 371 Date: May 5, 2017

Related U.S. Application Data
(60) Provisional application No. 62/075,973, filed on Nov. 6, 2014.

(51) Int. Cl. A61K 31/353; A61K 9/00; A61K 45/06; G01N 33/68; C12Q 1/6876

(52) U.S. Cl. CPC A61K 31/353; A61K 9/0053; A61K 9/0019; A61K 48/00; G01N 33/6887; C12Q 1/6876; A61K 45/06

(57) ABSTRACT

Processes for increasing expression of a MTOR gene or a mTOR protein or decreasing expression of a MSTN gene or a myostatin protein in a subject in need of such expression changes are provided. The processes include administering to the subject a composition including at least 0.5% Type-A polymers by weight. The Type-A polymers include A-Type doubly-linked procyanidin oligomers of the catechins and/or epicatechins. The processes further include increasing expression of a MTOR gene or a mTOR protein or decreasing expression of a MSTN gene or a myostatin protein in the subject by the step of administering.

### CROSS REFERENCE TO RELATED APPLICATIONS

This application depends from and claims priority to U.S. Provisional Application No. 62/075,973 filed Nov. 6, 2014, the entire contents of which are incorporated herein by reference.

### FIELD

The present disclosure relates to the use of Type A polymers, including A-Type doubly linked procyanidin oligomers of the catechins and/or epicatechins, and methods for promoting enhanced expression of a MTOR gene or a mTOR protein or decreased expression of a MSTN gene or a myostatin protein, or any combination thereof, in skeletal muscle.

### BACKGROUND

Dysregulated cellular function underlies many pathological conditions. Identification of molecular effectors of proper cell function along with methods of preventing or reversing cell dysfunction are required for promoting or maintaining proper or enhanced cellular function. Dysregulated gene expression is implicated in a variety of diseases such as cancer, diabetes, obesity, cardiovascular diseases and neurodegeneration. Discerning which molecular targets are most directly associated with a disease or condition is paramount to treating or preventing the disease and condition.

mTOR (mammalian target of rapamycin, also called mechanistic target of rapamycin and FRAP) is a serine/threonine protein kinase that is involved in a multitude of cellular processes, including regulation of cell growth and survival, cell metabolism, cell motility, cell proliferation, protein synthesis and transcription. mTOR mediates cellular responses to hormones, growth factors and nutrients, such as amino acids, as well as various stressors such as nutrient deprivation and hypoxia.

mTOR is a key regulator for muscle synthesis. Dysregulated mTOR is implicated in skeletal muscle dysfunction such as atrophy and in diseases such as muscular dystrophy.

Activated mTOR up-regulates protein synthesis by phosphorylating key regulators of mRNA translation and ribosome synthesis. Leucine is known to activate mTOR, “switching on” muscle building.

Other genes have been shown to correlate to muscle growth or lack thereof. Myostatin, or growth/differentiation factor 8 (GDF-8), belongs to the transforming growth factor-β (TGF-β) superfamily. The human myostatin protein is known to be expressed in skeletal muscle. Myostatin immunoreactivity is detectable in human skeletal muscle in both type 1 and type 2 fibers.

Myostatin levels may play a key role in negatively regulating muscle development. In the myostatin null mice, animals are substantially normal except that they are significantly larger than wild-type mice and have a large and widespread increase in skeletal muscle mass. Other organisms are affected by myostatin function, For example, two breeds of cattle, characterized by increased muscle mass, have mutations in the myostatin coding sequence (McPherron et al., Proc. Natl. Acad. Sci. 94:12457-61 (1997)). Additionally, the serum and intramuscular concentrations of immunoreactive myostatin are increased in HIV-infected men with muscle wasting compared with healthy men, and correlate inversely with the fat-free mass index. These data support the hypothesis that myostatin is a negative regulator of skeletal muscle growth in adult men and contributes to muscle wasting in HIV-infected men.

As such there exists a need for compositions and methods of increasing expression of a MTOR gene or a mTOR protein or decreasing expression of a MSTN gene or a myostatin protein in skeletal muscle cells.

### SUMMARY

It is understood that both the following summary and the detailed description are exemplary and explanatory and are intended to provide further explanation of the disclosure as claimed. Neither the summary nor the description that follows is intended to define or limit the scope of the disclosure to the particular features mentioned in the summary or description.

One object is to provide a method for altering intracellular levels of MTOR gene expression product(s) or a mTOR protein or decreasing expression of a MSTN gene product(s) or a myostatin protein in skeletal muscle cells of a subject. Such expression can be used restore abnormal expression of these expression products in a subject.

This object is achieved in the present disclosure that provides processes for increasing expression of a MTOR gene or a mTOR protein or decreasing expression of a MSTN gene or a myostatin protein in a subject. The processes include administering to the subject a composition comprising at least 0.5% Type-A polymers by weight. The Type-A polymers include A-Type doubly linked procyanidin oligomers of the catechins and/or epicatechins. Further processes include increasing expression of a MTOR gene or a mTOR protein or decreasing expression of a MSTN gene or a myostatin protein in said subject by the step of administering.

### DETAILED DESCRIPTION

The following description of particular aspect(s) is merely exemplary in nature and is in no way intended to limit the scope of the invention, its application, or uses, which may, of course, vary. The disclosure is described with relation to the non-limiting definitions and terminology included herein. These definitions and terminology are not designed to function as a limitation on the scope or practice of the disclosure but are presented for illustrative and descriptive purposes only.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

The present disclosure advantageously has utility as a composition that increases expression of a MTOR gene or a mTOR protein or decreases expression of a MSTN gene or a myostatin protein in skeletal muscle cells. Compositions and processes are provided that increase expression of a MTOR gene or a mTOR protein or decrease expression of a MSTN gene or a myostatin protein in skeletal muscle cells and are useful for enhancing cell and organism vitality. The disclosure provides materials in the form of Type-A polymers including A-Type singly or doubly linked procyanidin oligomers of the catechins or epicatechins that have utility for altering the expression level of myostatin and mTOR genes or proteins in skeletal muscle cells in subjects.

The inventors unexpectedly discovered that administration of a therapeutically effective amount of Type-A polymers as described herein or their equivalents functions in a subject to enhance expression level or rate of one or more genes that encode a mTOR (such as MTOR) or increases in levels of a mTOR protein as well as inhibit expression level or rate of one or more genes that encode myostatin (such as MSTN) or decreases in levels of a myostatin protein in skeletal muscle cells.

Processes are provided for enhancing expression of a MTOR gene or a mTOR protein or inhibiting expression of a MSTN gene or a myostatin protein in a subject. As used herein, a “subject” is defined as an organism (such as a human, non-human primate, equine, bovine, murine, or other mammal), or a cell. Illustrative examples of cells include skeletal muscle cells, such as L6 skeletal muscle cells. As used herein, a subject in need is defined as a subject that has decreased levels of MTOR gene or a mTOR protein relative to normal levels and/or increased levels of a MSTN gene or a myostatin protein relative to normal levels, or that desires to have increased levels of MTOR gene or a mTOR protein relative to normal levels and/or decreased levels of a MSTN gene or a myostatin protein relative to normal levels or the subject's own baseline levels.

Processes provided include administering to a subject a composition that includes one or more Type-A polymers. A composition optionally includes at least 0.5% Type-A polymers by dry weight. Type-A polymers optionally include A-Type singly and/or doubly linked procyanidin oligomers of the catechins and/or epicatechins. Such A-Type singly linked procyanidin oligomers of the catechins and/or epicatechins can include A-Type singly linked procyanidin dimers, A-Type singly linked procyanidin trimers, A-Type singly linked procyanidin trimers, A-Type singly linked procyanidin tetramers, and/or a mixture of A-Type singly linked procyanidin oligomers. Such A-Type doubly linked procyanidin oligomers of the catechins and/or epicatechins can include A-Type doubly linked procyanidin dimers, A-Type doubly linked procyanidin trimers, A-Type doubly linked procyanidin trimers, A-Type doubly linked procyanidin tetramers, and/or a mixture of A-Type doubly linked procyanidin oligomers.

The term “enhancing” as used herein is defined as an increase in expression, activity, or effect relative to a control related to the presence of an effector such as a Type-A polymer or a component thereof, such as A-Type doubly linked procyanidin oligomers of the catechins and/or epicatechins. In certain aspects, such A-Type doubly linked procyanidin oligomers of the catechins and/or epicatechins can include A-Type singly and doubly linked procyanidin dimers, A-Type singly and doubly linked procyanidin trimers, A-Type singly and doubly linked procyanidin tetramers, and/or a mixture of A-Type singly and doubly linked procyanidin oligomers. In other aspects, such A-Type doubly linked procyanidin oligomers of the catechins and/or epicatechins can include A-Type doubly linked procyanidin dimers, A-Type doubly linked procyanidin trimers, A-Type doubly linked procyanidin tetramers, and/or a mixture of A-Type doubly linked procyanidin oligomers. Illustrative examples of “enhanced” are increases in the expression level or rate of one or more genes that encode a mTOR such as MTOR, or increases in levels of a mTOR protein.

The term “inhibiting” is defined as a decrease in expression, activity, or effect relative to a control related to the presence of an effector such as a Type-A polymer or a component thereof, such as A-Type doubly linked procyanidin oligomers of the catechins and/or epicatechins. In certain aspects, such A-Type doubly linked procyanidin oligomers of the catechins and/or epicatechins can include A-Type singly and doubly linked procyanidin dimers, A-Type singly and doubly linked procyanidin trimers, A-Type singly and doubly linked procyanidin tetramers, and/or a mixture of A-Type singly and doubly linked procyanidin oligomers. In other aspects, such A-Type doubly linked procyanidin oligomers of the catechins and/or epicatechins can include A-Type doubly linked procyanidin dimers, A-Type doubly linked procyanidin trimers, A-Type doubly linked procyanidin tetramers, and/or a mixture of A-Type doubly linked procyanidin oligomers. Illustrative examples of inhibiting are decreases in the expression level or rate of one or more genes that encode myostatin such as MSTN, or decreases in levels of a myostatin protein.

“Active ingredient” refers a component present in the composition that renders, directly or indirectly, the intended effect. One particular example is a polyphenol type-A polymer, with more particular examples being singly linked type-A polymers and/or doubly linked type-A polymers. Thus, in some aspects, an “active ingredient” can include A-Type singly or doubly linked procyanidin dimers of catechins and/or epicatechins, A-Type singly or doubly linked procyanidin trimers of catechins and/or epicatechins, A-Type singly or doubly linked procyanidin tetramers of catechins and/or epicatechins, and/or a mixture of A-Type singly or doubly linked procyanidin oligomers of catechins and/or epicatechins. In other aspects, an “active ingredient” can include A-Type doubly linked procyanidin dimers of catechins and/or epicatechins, A-Type doubly linked procyanidin trimers of catechins and/or epicatechins, A-Type doubly linked procyanidin tetramers of catechins and/or epicatechins, and/or a mixture of A-Type doubly linked procyanidin oligomers of catechins and/or epicatechins. In some aspects, the term “active ingredient” excludes singly linked Type-A polymers.

“Polyphenol” as used herein refers to a group of chemical substances found in plants, characterized by the presence of more than one phenol group per molecule. For purposes of this disclosure, it is to be understood that polyphenols include, but are not limited to, Type-A polymers and oligomers or phenolic materials. Natural sources of polyphenols include green tea, white tea, red wine, dark chocolate, olive oil, and other fruits, vegetables, and plants including cinnamon.

Type-A polymers as used herein are a bioactive type of naturally available polymers. They are identified by their protonated molecular masses as A-type singly or doubly linked procyanidin oligomers of the catechins and/or epicatechins. The polymers are composed of monomeric units of catechins and/or epicatechins with a molecular mass of 288 Da. A-type doubly linked procyanidin oligomers may have masses ranging from 576 to 1728 Da and may include dimers, trimers, tetramers, and a mixture of oligomers, respectively. For example, two separate doubly linked Type A trimers and a doubly linked Type A tetramer have molecular masses of 864 and 1152 Da, respectively. The trimer and tetramer oligomers include terminal (T), middle (M) and base (B) units, with the M unit of the two trimers consisting of a single catechin/epicatechin and the M unit of the tetramer consisting of two catechins/epicatechins. Doubly linked procyanidin type-A oligomers of the catechins and/or epicatechins contain C4→C8 carbon and C2→O7 ether bonds between the T and M units of the oligomers, and have the structure:

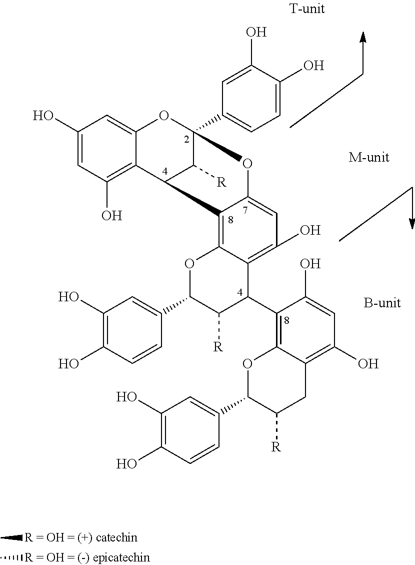

(Anderson et al., J. Agric. Food Chem., 2004; 52:65-70.) Thus, in certain aspects of the present disclosure Type-A polymers can include A-type singly or doubly linked procyanidin dimers of catechins and/or epicatechins, A-type singly or doubly linked procyanidin trimers of catechins and/or epicatechins, A-Type singly or doubly linked procyanidin tetramers of catechins and/or epicatechins, and/or a mixture of A-Type singly or doubly linked procyanidin oligomers of catechins and/or epicatechins. In other aspects, Type-A polymers can include A-Type doubly linked procyanidin dimers of catechins and/or epicatechins, A-type doubly linked procyanidin trimers of catechins and/or epicatechins, A-Type doubly linked procyanidin tetramers of catechins and/or epicatechins, and/or a mixture of A-Type doubly linked procyanidin oligomers of catechins and/or epicatechins. In some aspects, A-Type singly linked procyanidin oligomers of the catechins and/or epicatechins are excluded from a composition.

In some aspects, any composition containing at least 0.5% Type-A polymers by dry weight may be utilized for administration to a subject such as compositions included in a dietary supplement. The amount of polyphenol Type-A polymers or the like is optionally in the range of 0.5% to 25%, optionally 1% to 10% by weight. Optionally, the amount of polyphenol Type-A polymers is at or greater than 0.5%, at or greater than 1%, at or greater than 2%, at or greater than 3%, at or greater than 4%, at or greater than 5%, or at or greater than 10% by weight. The amount of doubly-linked polyphenol Type-A polymers or the like is optionally in the range of 0.5% to 25%, optionally 1% to 10% by weight. Optionally, the amount of doubly-linked polyphenol Type-A polymers is at or greater than 0.5%, at or greater than 1%, at or greater than 2%, at or greater than 3%, at or greater than 4%, at or greater than 5%, or at or greater than 10% by weight. In some aspects, a Type-A polymer is or is a part of a dietary supplement composition. A Type-A polymer is optionally present in a dietary supplement composition at 0.5%-100% by weight, optionally 1%-50% by weight, optionally 1%-10% by weight, optionally 20%-50% by weight, optionally 30%-40% by weight, or any value or range between 0.5% and 100% of the dry weight of the dietary supplement composition.

In an exemplary regimen, the compositions are taken orally between one and three times daily; although, other routes of administration may be utilized. Also, it should be noted that the compositions may be utilized in the form of derivatives. For example, the composition may be bonded, chemically or physically, to other chemical species and moieties such as synthetic polymers, liposomes, small organic molecules, chitin, chitosan, other biopolymers and the like. In view of the teaching presented herein, still further combinations will be readily apparent to those of skill in the art.

In some aspects, a subject is administered a composition in a dosage so that each dose of the compositions is selected to deliver into the Type-A polymers in the amount of 0.1 milligrams (mg) to 150 mg of Type-A Polymer per serving or any value or range therebetween, optionally 1-30 mg, optionally 3-10 mg. It is further contemplated that variable dosing regiments are operative in the processes. While in some instances, a single dose treatment may be effective in producing therapeutic or other desired effects, in other instances a treatment period in the range of, for example, six weeks to three or six months or more may be utilized.

A composition may be administered orally, parentally, or intravenously by intramuscular, intraperitoneally, by transdermal injection, or by contact with a cell or tissue such as by immersion or other form of contact. Injectables or oral dosage forms may be prepared in conventional forms, either liquid solutions or suspensions, solid forms suitable for solution or prior to administration, or as suspension in liquid prior to administration or as emulsions.

The dose of the composition may vary depending on the age, weight, general condition of the subject. For example, dosage in the range of 1-1,000 mg or equivalent of at least 0.5% Type-A polymers by dry weight per day may be an effective range. The active agent be present at 0.01%-100% of the dry weight of the composition. For example, an active agent may comprise 0.5%-50% of the dry weight of the composition.

Administration of a composition to a subject will inhibit expression of a myostatin gene or protein in a subject relative to control or baseline. Illustratively, a myostatin gene expression (such as MSTN) is decreased as measured by the level of myostatin mRNA relative to a control such as the absence of composition. Illustratively, myostatin gene expression is inhibited (e.g. decreased) by a value of 1% to 300% or more, or any value or range therebetween. Optionally, myostatin gene expression is decreased by 1%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 250%, 300%, or more.

The level of target of rapamycin (mTOR) gene expression (such as MTOR) may be enhanced (e.g. increased) in a subject relative to control or baseline. Illustratively, expression of the gene encoding mTOR (MTOR) is enhanced by a value of 1% to 300% or more, or any value or range therebetween. Optionally, expression of the gene encoding mTOR is enhanced by 1%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 250%, 300%, or more relative to control.

Methods for detecting mRNA expression to determine the presence or extent of gene expression are known in the art. Illustratively, mRNA is detected and optionally quantified by real-time polymerase chain reaction (qRT-PCR as used herein). qRT-PCR is optionally coupled to prior synthesis of cDNA from total cellular RNA such as using Superscript II RT which is a reverse transcriptase enzyme produced by Invitrogen, Corp., Carlsbad, Calif. Illustrative protocols for measuring gene expression can be found in Crujeiras A B, et al., Eur J Clin Invest, 2008; 38(9):672-8 as well as in other sources known in the art.

Expression of a mTOR protein in a subject is optionally enhanced (i.e. increased) by administration of a composition to a subject. Illustratively expression of the mTOR protein is enhanced relative to a control or baseline. Illustratively, mTOR protein expression is enhanced by a value of 1% to 300% or more, or any value or range therebetween. Optionally, mTOR protein expression is enhanced by 1%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 250%, 300%, or more.

Expression of a myostatin protein in a subject is optionally inhibited (i.e. decreased) by administration of a composition to a subject. Illustratively expression of the myostatin protein is inhibited relative to a control or baseline. Illustratively, myostatin protein expression is inhibited by a value of 1% to 300% or more, or any value or range therebetween. Optionally, myostatin protein expression is inhibited by 1%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 250%, 300%, or more.

Detecting and optionally quantifying myostatin or mTOR protein expression is achieved by many methods known in the art. Illustratively, myostatin or mTOR protein expression is detected and optionally quantified by enzyme linked immunosorbent assay (ELISA), mass spectrometry, western blot, gel electrophoresis optionally coupled with staining such as by Coomassie brilliant blue or silver stain, or by target specific stains, flow cytometry, immunoprecipitation, or by other method known in the art. In some aspects, an ELISA is used to detect and optionally quantify myostatin or mTOR protein expression. For example, ELISA kits for myostatin or mTOR are available from sources known in the art. Antibodies directed to myostatin or mTOR proteins suitable for use in ELISA are available from Santa Cruz Biotechnology, Santa Cruz, Calif.

The administered dose of the composition may vary depending on the age, weight, or general condition of the user. For example, dosage in the range of 1-1,000 mg or equivalent of at least 0.5% Type-A polymers by dry weight per day may be an effective range. The active agent may be present at 0.01%-100% of the dry weight of the composition.

It is appreciated that any dietary supplement or any extract of cinnamon described herein or their equivalents are optionally used in a process to enhance lean muscle mass or to alter glucose metabolism in a subject.

A process of increasing insulin sensitivity (i.e. glucose metabolism) of a subject is also provided. Such processes illustratively include administering to a subject a therapeutically effective amount of a composition including one or more Type-A polymers. A therapeutically effective amount is defined as that capable of increasing the expression of a mTOR protein or a gene encoding a mTOR protein (such as MST1V) or decreasing the expression of a myostatin protein or a gene encoding a myostatin protein (such as MTOR) relative to a control. A composition includes at least 0.5% Type-A polymers by dry weight. In some aspects, at least 0.5% Type-A polymers by dry weight may be included in a composition such as in a dietary supplement. As described above, Type-A polymers include polyphenol type-A polymers, with more particular examples being singly linked type-A polymers and/or doubly linked type-A polymers. In some aspects, Type-A polymers can include A-type singly or doubly linked procyanidin dimers of catechins and/or epicatechins, A-type singly or doubly linked procyanidin trimers of catechins and/or epicatechins, A-Type singly or doubly linked procyanidin tetramers of catechins and/or epicatechins, and/or a mixture of A-Type singly or doubly linked procyanidin oligomers of catechins and/or epicatechins. In other aspects, Type-A polymers can include A-type doubly linked procyanidin dimers of catechins and/or epicatechins, A-type doubly linked procyanidin trimers of catechins and/or epicatechins, A-type doubly linked procyanidin tetramers of catechins and/or epicatechins, and/or a mixture of A-type doubly linked procyanidin oligomers of catechins and/or epicatechins. Type-A polymers optionally are or are a part of a dietary supplement composition.

Processes of increasing insulin sensitivity illustratively include administering to a subject a dietary supplement composition including Type-A polymers in a dosage so that each dose of the composition will deliver into the individual Type-A polymers in the amount of 0.1 milligrams (mg) to 150 mg of Type-A Polymer per serving or any value or range therebetween, optionally 1-30 mg, optionally 3-10 mg. As described above, Type-A polymers include polyphenol type-A polymers, with more particular examples being singly linked type-A polymers and/or doubly linked type-A polymers. In some aspects, Type-A polymers can include A-type singly or doubly linked procyanidin dimers of catechins and/or epicatechins, A-type singly or doubly linked procyanidin trimers of catechins and/or epicatechins, A-Type singly or doubly linked procyanidin tetramers of catechins and/or epicatechins, and/or a mixture of A-Type singly or doubly linked procyanidin oligomers of catechins and/or epicatechins. In other aspects, Type-A polymers can include A-type doubly linked procyanidin dimers of catechins and/or epicatechins, A-type doubly linked procyanidin trimers of catechins and/or epicatechins, A-type doubly linked procyanidin tetramers of catechins and/or epicatechins, and/or a mixture of A-type doubly linked procyanidin oligomers of catechins and/or epicatechins. It is further contemplated that variable dosing regiments are operative in the methods. While in some instances, a single dose treatment may be effective in producing therapeutic effects, in other instances a treatment period in the range of, for example, six weeks to three or six months or more may be utilized. The composition may be administered orally, parentally, or intravenously by intramuscular, intraperitoneally, by transdermal injection, or otherwise by contact with a subject. Injectables may be prepared in conventional forms, either liquid solutions or suspensions, solid forms suitable for solution or prior to injection, or as suspension in liquid prior to injection or as emulsions.

The dose of the composition may vary depending on the age, weight, general condition of the subject. For example, dosage in the range of 1-1,000 mg or equivalent of at least 0.5% Type-A polymers by dry weight per day may be an effective range. The active agents may also comprise 0.01%-100% of the dry weight of the composition.

It is appreciated that any composition described herein or their equivalents are optionally used in a process to increase insulin sensitivity.

Insulin sensitivity is optionally measured by methods known in the art, illustratively by a hyperinsulinemic-euglycemic clamp technique (DeFronzo, R. A., et al., Am. J. Physiol. 237:E214-223, 197) or an oral or intravenous glucose tolerance test (Cutfield, W. S., et al., J. Clin. Endocrinol. Metab. 70:1644-1650, 1990). Other methods known in the art are similarly operable. Insulin sensitivity is optionally increased by 5% or more. Insulin sensitivity is optionally increased by 5% to 200% or more, or any value or range between 5% to 200%.

A process of increasing muscle mass of a subject is also provided. Such processes illustratively include administering to a subject a therapeutically effective amount of Type-A polymers. A therapeutically effective amount is defined as that capable of increasing the expression of a mTOR protein or a gene encoding a mTOR protein (such as MSTN) or decreasing the expression of a myostatin protein or a gene encoding a myostatin protein (such as MTOR) relative to a control. A composition includes at least 0.5% Type-A polymers by dry weight. In some aspects, any composition containing at least 0.5% Type-A polymers by dry weight may be included in a composition such as in a dietary supplement. As described above, Type-A polymers include polyphenol type-A polymers, with more particular examples being singly linked type-A polymers and/or doubly linked type-A polymers. In some aspects, Type-A polymers can include A-type singly or doubly linked procyanidin trimers of catechins and/or epicatechins, A-Type singly or doubly linked procyanidin tetramers of catechins and/or epicatechins, and/or a mixture of A-Type singly or doubly linked procyanidin oligomers of catechins and/or epicatechins. In other aspects, Type-A polymers can include A-type doubly linked procyanidin dimers of catechins and/or epicatechins, A-type doubly linked procyanidin trimers of catechins and/or epicatechins, A-type doubly linked procyanidin tetramers of catechins and/or epicatechins, and/or a mixture of A-type doubly linked procyanidin oligomers of catechins and/or epicatechins. A composition optionally is or is a part of a dietary supplement composition. A composition is optionally present in a dietary supplement composition at 0.5%-100% by weight, optionally 20%-50% by weight, optionally 30%-40% by weight, or any value or range between 10% and 100% of the dry weight of the dietary supplement composition.

Processes of increasing muscle mass illustratively include administering to a subject a composition including Type-A polymers in a dosage so that each dose of the composition will deliver into the individual Type-A polymers in the amount of 0.1 milligrams (mg) to 150 mg of Type-A Polymer per serving or any value or range therebetween, optionally 1-30 mg, optionally 3-10 mg. As described above, Type-A polymers include polyphenol type-A polymers, with more particular examples being singly linked type-A polymers and/or doubly linked type-A polymers. In some aspects, Type-A polymers can include A-type singly or doubly linked procyanidin dimers of catechins and/or epicatechins, A-type singly or doubly linked procyanidin trimers of catechins and/or epicatechins, A-Type singly or doubly linked procyanidin tetramers of catechins and/or epicatechins, and/or a mixture of A-Type singly or doubly linked procyanidin oligomers of catechins and/or epicatechins. In other aspects, Type-A polymers can include A-type doubly linked procyanidin dimers of catechins and/or epicatechins, A-type doubly linked procyanidin trimers of catechins and/or epicatechins, A-type doubly linked procyanidin tetramers of catechins and/or epicatechins, and/or a mixture of A-type doubly linked procyanidin oligomers of catechins and/or epicatechins. It is further contemplated that variable dosing regiments are operative in the methods. While in some instances, a single dose treatment may be effective in producing therapeutic effects, in other instances a treatment period in the range of, for example, six weeks to three or six months or more may be utilized. The composition may be administered orally, parentally, or intravenously by intramuscular, intraperitoneal, by transdermal injection, or otherwise by contact with a subject. Injectables or oral forms may be prepared in conventional forms, either liquid solutions or suspensions, solid forms suitable for solution or prior to administration, or as suspension in liquid prior to administration or as emulsions.

The dose of the composition may vary depending on the age, weight, general condition of the subject. For example, dosage in the range of 1-1,000 mg of at least 0.5% Type-A polymers by dry weight per day may be an effective range. The Type-A polymers may also comprise 0.01%-100% of the dry weight of the composition. For example, a dietary supplement composition may comprise 0.5%-50% of the dry weight of the composition.

Muscle mass is optionally measured by methods known in the art. Muscle mass is optionally increased by 0.1% or more.

A composition is provided that includes Type-A polymers such as A-Type singly and/or doubly linked procyanidin oligomers of the catechins and/or epicatechins, and may be in dietary supplement compositions in solid, semi-solid, or liquid dosage forms, such as, for example, tablets, chewables, suppositories, pills, capsules, powders, liquids, or suspensions, and may be provided in unit dosages suitable for a single administration. Time release preparations are also contemplated as effective dosage formulations. The compositions may include an effective amount of the Type-A polymers alone or in combination with a pharmaceutically acceptable carrier and, in addition, may include other medicinal agents, pharmaceutical agents, carriers, or diluents.

Type-A polymers such as A-Type singly and/or doubly linked procyanidin oligomers of the catechins and/or epicatechins may be chemically synthesized or obtained from one or more natural sources. One or more of any process for isolating chemical material from a plant may be used to isolate the Type-A polymers such as A-Type singly and/or doubly linked procyanidin oligomers of the catechins and/or epicatechins. One exemplary source of type-A polymers is cinnamon. Cinnamon may be obtained from various resources. Illustratively, cinnamon is obtained from bark. Cinnamon bark may be obtained from various parts of the world, including China, Sri Lanka, Indonesia and others. The type-A polymers are optionally obtained by carefully tailored extraction procedures from cinnamon bark. An extract of cinnamon is optionally derived from any Cinnamonum species. In an exemplary aspect, an extract of cinnamon is derived from the bark of Cinnamonum aromaticum, Cinnamonum verum, or Cinnamomum burmannii. In some aspects, an extract of cinnamon is derived from the bark of the Cinnamomum zeylanicum tree of the genus Lauraceae. This tree is native to eastern and southeastern Asia. Other sources of cinnamon may also be used in the methods and materials disclosed herein. Cinnamon bark may be used in the form of raw bark, sliced, or minced bark, or pulverized bark for the preparation of the therapeutic materials, and pulverized cinnamon bark is used in particular instances.

Extracts may be prepared by various methods carefully tailored to produce the required concentration or amount of type-A polymers. The extracts are optionally water soluble. As such, the extracts are optionally water soluble water extracts, water soluble alcohol extracts, or water soluble extracts of other operative extraction processes. The extraction process is directly linked to the final composition of the resulting extract. As such, a product formed by one process does not necessarily equate to an extract formed by a different process, often differing by a single extraction parameter. The processes described herein represent exemplary methods to produce extracts with the desired level of the active agent—Type-A polyphenols, particularly doubly linked Type-A polymers.

Extraction parameters such as water quality, heating temperature, drying temperature, heating time, drying time, and filtering processes all contribute to the quality and efficiency of the processes. Water quality directly affects the concentration of active agents. Poor quality water may cause type-A polymers to become decomposed and oxidized during the extraction process. This often results in cinnamon extract powder being reddish in color and the percent concentration of type-A polymers being low. Heating time determines the ratio of various polymers being extracted. Heating time also affects the thickness of extraction mixture which then has a direct impact on the downstream filtering process. The temperature of the extraction also affects the level of active type-A polymers. In some aspects, the extraction temperature is between 50° C. and 100° C. Optionally, the extractions temperature is between 50° C. and 95° C. Optionally, the temperature is between 50° C. and 90° C. Optionally the extraction temperature is between 50° C. and 90° C. Drying temperature may vary from 75° C. to 120° C. depending on what other extraction parameters are also used. The amount of solvent used is generally from 2 to 100 times the raw extract material on a weight basis. Illustratively, when 50 g of cinnamon bark is used, the extraction is performed with 1000 ml of water (1 g/ml is weight of water—i.e. 20 times volume).

Extraction time is also important for obtaining the desired amount of, polyphenol Type-A polymers, which are described in detail above. Extractions are optionally performed by heating the raw material in an extraction solvent in excess of 10 minutes, optionally, in excess of 1 hour, optionally between 1 and 3 hours with any subdivision also operable.

Extraction solvents are optionally aqueous or organic. Distilled water or alcohols such as ethanol are optionally used alone or in combination as extraction solvents. The extracts obtained are optionally water soluble.

Illustrative examples of cinnamon extracts that contain the requisite amount of type-A polymers are found in U.S. Pat. No. 6,200,569, and therein describing the product sold as CINNULIN PF.

In some aspects, 50 g clean cinnamon bark is ground into small particles or powder. The powder or particles are mixed with 1000 ml distilled water in a suitable flask. The mixture is let stand at room temperature for about 0.5 hour. In this and other examples, an amount of buffer is optionally added to maintain the pH of the extraction solvent. Additional water may be added is in the range of 1:20 to 1:2000. Too little water may render the mixture too thick for extraction. However, too much water increases drying time. Then the water mixture is heated while being stirred through the use of a magnetic heat stirrer. The temperature and extraction time are crucial to the concentration efficiency of the bioactive polymers. The extraction process is optionally no longer than one hour. Optionally, the ground bark may be heated for 15-20 minutes bringing to a boil, simmering for 20-30 minutes while stirring constantly. Optionally, the ground bark is heated to 100° C. 15-20 minutes and then simmered for 20-30 minutes while stirring constantly. The boiling time is optionally controlled at about 20-25 minutes following heating. The mixture is cooled and stored at 4° C. overnight. An exemplary cinnamon extract obtained by a water extraction is sold as CINNULIN PF.

In one exemplary aspect, 250 kg of Cinnamomum burmannii, is ground into small particles or powder. The powder or particles are mixed with 2000 ml (8×) distilled ethanol-water in a suitable flask and the mixture is allowed to stand at ambient temperature for 0.5 hours. Optionally, water alone is used as the extraction solvent illustratively by using a 10× fold-water volume/weight ground cinnamon bark. The mixture is heated to 50° C. while being stirred through the use of a magnetic heat stirrer and circulated for 120 min. Evaporation is performed at a steam temperature of less than 100° C. with a process temperature of less than 60° C. with a TS refract meter of 45-50%. The material is then dried to a moisture content of less than 5%.

In some exemplary aspects, Type-A polyphenols are extracted from cinnamon using the following process: 5 g cinnamon and 100 ml 0.1 N acetic acid are combined and autoclaved for 15 minutes. The resultant mixture is cooled, then centrifuged and the precipitate discarded. Four volumes of ethanol/0.1 N acetic acid are added to the supernatant and the mixture is stored overnight at 4 C°. The mixture is screened through a filter. To determine the amount of bioactive polymers the mixture is introduced onto an LH-20 column and washed with 600 ml ethanol/0.1 N acetic acid. The desired fraction is then eluted with a 1:1 mixture of acetonitrile and 0.2 N acetic acid. The eluate is then concentrated and introduced onto a HPLC column at 275 nm.

In some aspects, the initial extraction is performed in the absence of acid. 50 g clean cinnamon bark is ground into small particles or powder and mixed with 1000 ml distilled water/10% ethanol in a suitable flask. Then the water mixture is heated while being stirred through the use of a magnetic heat stirrer. The extraction process is optionally no longer than one hour. Optionally, the ground bark in extraction solvent is heated to a boil for 15-20 minutes, and then simmered for 20-30 minutes while stirring constantly. The boiling time is typically controlled at about 20-25 minutes following heating. The mixture is cooled and stored at 4° C. overnight. It is appreciated that alcohols other than or in addition to ethanol, illustratively methanol, may be used in the extraction procedure as well. When alcohol is used in the extraction solvent is it generally present at 50% or less.

Any one of the extraction solutions (or combinations thereof) described herein is optionally filtered through a filter paper to remove any solid debris. If the solution is too thick for the filter paper, the removal of solids from the solution is optionally done with the use of centrifugation. The resulting supernatant is filtered through medium speed filter paper. The resulting solids are optionally dissolved in 200 mL distilled water, or water/ethanol for a second extraction. The liquid solution containing the solids is mixed and heated for 30 minutes at 80-90° C. and then is filtered to produce a second extraction solution.

In some aspects, first and second extraction solutions are combined together and poured onto nonstick tray and allowed to dry at 80-90° C. Vacuum-spray dry equipment is optionally used for the drying procedure. The resulting dry extract powder is weighed. An extraction ratio is calculated as w/20×100% with w as the weight (g) of the dry extract powder. The sample and water ratio, heat time, volume of water in the second extraction may vary depending on the amount of the raw material used for extraction.

High performance liquid chromatography (HPLC) is optionally employed to analyze the effect on the concentrations of the polymers by changes in heating temperature and extraction time. As a non-limiting example, 100 mg dry cinnamon powder is dissolved with 100 ml water in a flask. The solution is sonicated for 30-45 minutes and filtered through 0.45 μm PTFE syringe. The samples are prepared and tested at different temperatures as follows: samples are extracted at 50-60° C. for one hour, Type-A polymers eluting at 17 and 21 minutes have reasonable concentrations. After increasing the temperature to 75-82° C. for 1 hour, the peaks eluting at 17 and 21 minutes are decreased by 2-3%. There are additional two relatively small peaks that seem to surface during this extraction. They elute at 28.5 minutes, 33.5 minutes respectively. After the heating temperature is increased to 85-90° C. for an additional 1 hour, the peaks eluting at 17 and 21 minutes are decreased about 7-9%. The peaks at 28.5 and 33.5 increase significantly. Lastly, the heating temperature is increased to 95-100° C. for 20 minutes and then reduced to 85-95° C. for an additional 40 minutes. The peaks eluting at 17 and 21 minutes seem to decrease by 15-20%. The peaks eluting at 28.5 and 33.5 minutes increase by more than double. According to these results, the polymers at 17 and 21 minutes are converted to isomers at 28.5 and 33.5 minutes respectively.

In another procedure, the stabilization of the Type-A polymers is analyzed. Various extraction periods at heating temperature of 50-100° C. are tested particularly 95-100° C. After samples are extracted at 50-100° C. for one hour, polymer eluting at 17 and 21 minutes presents desirable concentrations. The peaks eluting at 17 and 21 minutes decrease as the heating temperature increases in the first 2-3 hours. After 3 hours, the peaks eluting at 17 and 21 minutes no longer change as significantly and seem to reach a plateau period. These results suggest that after a 3 hour extraction time at temperature of 95-100° C., polymers are stabilized.

Not only is it important to note that the time and temperature play a key factor in sustaining higher concentrations of these Type-A polymer key actives, additionally the species of choice can have a dramatic impact on the levels of these Type-A polymers. After thorough review of the world's many species of cinnamon, the following has proven to provide the highest level of active Type-A polymers: Cinnamomum Burmannii (Nees) Blume—Microbial Identification Index (MIDI) class; Korintji Cassia.

Cinnamon extract dry powder prepared as discussed above is tested to confirm the presence of certain amount of polyphenols such as double-linked polyphenol Type-A polymers (which may include A-Type doubly linked procyanidin dimers of catechins and/or epicatechins, A-Type doubly linked procyanidin trimers of catechins and/or epicatechins, A-Type doubly linked procyanidin tetramers of catechins and/or epicatechins, and/or a mixture of A-Type doubly linked procyanidin oligomers of catechins and/or epicatechins), singly-linked Type-A polymers (which may include A-Type singly linked procyanidin dimers of catechins and/or epicatechins, A-Type singly linked procyanidin trimers of catechins and/or epicatechins, A-Type singly linked procyanidin tetramers of catechins and/or epicatechins, and/or a mixture of A-Type singly linked procyanidin oligomers of catechins and/or epicatechins), or other bioactive polymers through the use of HPLC. This allows for standardization of the extract.

In particular instances, the dry weight of the cinnamon extract powder can be standardized on the basis of a bioactive component, such as doubly-linked polyphenol Type-A polymers. As described above, doubly-linked polyphenol Type-A polymers may include A-Type doubly linked procyanidin dimers of catechins and/or epicatechins, A-Type doubly linked procyanidin trimers of catechins and/or epicatechins, A-Type doubly linked procyanidin tetramers of catechins and/or epicatechins, and/or a mixture of A-Type doubly linked procyanidin oligomers of catechins and/or epicatechins. The amount of polyphenol Type-A polymers or the like is optionally in the range of 0.5% to 25%, optionally 1% to 10% by weight. Optionally, the amount of polyphenol Type-A polymers is greater than 0.5%, greater than 1%, greater than 2%, greater than 3%, greater than 4%, greater than 5%, or greater than 10% by weight. In further aspects, the amount of doubly-linked polyphenol Type-A polymers or the like is optionally in the range of 0.5% to 25%, optionally 1% to 10% by weight. Optionally, the amount of doubly-linked polyphenol Type-A polymers is greater than 0.5%, greater than 1%, greater than 2%, greater than 3%, greater than 4%, greater than 5%, or greater than 10% by weight.

Depending on the source material, extraction procedures, extraction solvents, etc., the final concentration of Type-A polymers is often insufficient or less than 0.5%, less than 1%, less than 2%, less than 3%, less than 4%, less than 5%, or less than 10% by weight. As such, the extract may be further processed to concentrate the type-A polymers to the desired or necessary concentration. A liquid extract is optionally passed over a column to provide a concentrated eluant with the target concentration of type-A polymers.

Cinnamon bark may be used in the form of raw bark, sliced, or minced bark, or pulverized bark for the preparation of the therapeutic materials, and pulverized cinnamon bark is used in particular instances.

In one experimental series, an extract is prepared according to the foregoing procedures using a water extraction solvent. The concentration of the sample is approximately (e.g. within error) 5.17 mg/ml. It is also very important to note that the concentrations of the polymers change with the temperature and extraction time.

Depending on the intended mode of administration, the composition administered can be in pharmaceutical compositions in the form of solid, semi-solid or liquid dosage forms, such as, for example, tablets, suppositories, pills, capsules, powders, liquids, or suspensions, and may be provided in unit dosages suitable for a single administration. Time release preparations are specifically contemplated as effective dosage formulations. The compositions will include an effective amount of the selected substrate in combination with a pharmaceutically acceptable carrier and, in addition, may include other medicinal agents, pharmaceutical agents, carriers, or diluents.

In a solid composition aspect, conventional nontoxic solid carriers may include, for example, pharmaceutical grades of mannitol, lactose, starch, magnesium stearate, sodium saccharine, talc, cellulose, glucose, sucrose and magnesium carbonate. Liquid pharmaceutically administrable compositions may, for example, be prepared by dissolving or dispersing an active agent with optimal pharmaceutical adjuvants in an excipient, such as water, saline, aqueous dextrose, glycerol, or ethanol, to form a solution or suspension. For example, the pharmaceutical composition may contain minor amounts of nontoxic auxiliary substances such as wetting or emulsifying agents, pH buffering agents, for example, sodium acetate or triethanolamine oleate. Actual methods of preparing such dosage forms are known, or will be apparent, to those skilled in this art; for example, see Remington's The Science and Practice of Pharmacy (20^{th} Edition).

In oral administration aspects, fine powders or granules may contain diluting, dispersing, or surface active agents. The fine powders or granules may be presented in water or in syrup, in capsules or sachets in the dry state, or in a non-aqueous solution or suspension. Suspending agents may also be included in tablets, which may include binders and lubricants in a suspension. Flavoring, preserving, suspending, thickening, or emulsifying agents may be also included to modify the taste and texture of the composition. The tablets and granules provided for oral administration may further be coated for ease of digestion.

In some aspects, the composition containing the active Type-A polymers may be combined with one or more supplementary active agents. A supplementary active agent optionally functions synergistically with a Type-A polymer. Supplementary active agents illustratively include vitamins (such as vitamin A, vitamin B, vitamin C, vitamin D, vitamin E or vitamin K), antioxidants (such as acai, wolfberry, alpha lipoic acid, astazanthin, or fucoxanthin), other mTOR enhancers (illustratively branched chain amino acids, including leucine, isoleucine, valine, or combinations thereof), myostatin inhibitors (illustratively leucine, β-hydroxy β-methylbutyric acid, creatine, or combinations thereof) or any combination of the above.

The composition including Type-A polymers is optionally in the form of a food additive. Examples include foods in a liquid, semi-liquid, solid, paste, or jelly form.

Compositions are optionally metabolized in the subject to yield a therapeutically effective amount of compound species, namely polyphenols such as a Type-A polyphenols as discussed in detail above, cinnamon oligomer, cinnamon catechin or epicatechin, cinnamon chalcone, and cinnamon MHCP. In particular therapies, each dose of the cinnamon extract supplement is selected so as to deliver into the individual Type-A polymers in the amount of 0.1 milligrams (mg) to 150 mg of Type-A polymer per serving or any value or range therebetween, optionally 1-30 mg, and optionally 3-10 mg.

Various aspects of the present invention are illustrated by the following non-limiting examples. The examples are for illustrative purposes and are not a limitation on any practice of the present invention. It will be understood that variations and modifications can be made without departing from the spirit and scope of the invention.

### EXAMPLES

In the present study, human myoblasts and rat L6 skeletal muscle cells were used to determine the effects of active agents. More specifically immunofluorescence studies were utilized to explore the effects of an active composition including Type-A polymers on muscle mass related key proteins. Doubly linked procyanidin type-A polymers (3% by weight) in a dried extract of bark from C. burmanni was prepared as described (Anderson et al., J. Agric. Food Chem. 2004; 52:65-70), or provided by IN Ingredients Inc. (formerly Integrity Nutraceuticals, Columbia, Tenn., USA) as CINNULIN PF. The dried extract composition was solvated in DMSO and stored at −20° C. until use. The antibodies (anti-myostatin, anti-mTOR) were all obtained from Abcam.com (Cambridge, Mass., USA). All other reagents used were of the highest grade available in commercial products.

Human myoblasts were obtained from Gibco® Invitrogen, Life Technologies (Grand Island, N.Y., USA) and were maintained in DMEM medium supplemented with 2% horse serum in a humidified atmosphere containing 5% CO₂ and 95% air at 37° C. Cells were subcultured by trypsinization of subconfluent cultures using 0.05% trypsin with EDTA. Myoblasts were transferred to collagen I coated 35 mm dishes at a density of 5×10⁴ cells per dish. Myotube differentiation was promoted by substituting the proliferation media with DMEM-F12 Glutamax containing 2% FBS, 25 pM Insulin (Sigma-Aldrich, MO, USA) and 1% penicillin-streptomycin-glutamine. The myoblasts were cultured for two days. Then cells were incubated in fresh DMEM culture medium supplemented with the concentrations of extract of 5 μg/ml, 10 μg/ml, or 20 μg/ml for 24 h at 37° C.

Rat L6 myoblasts (CRL-1458) were purchased from ATCC, Manassas, Va. and were maintained in DMEM medium supplemented with 10% fetal bovine serum and 1% penicillin-streptomycin-glutamine in a humidified atmosphere containing 5% CO₂ and 95% air at 37° C. Cells were subcultured by trypsinization of subconfluent cultures using 0.05% trypsin with EDTA. L6 myoblasts were seeded at a density of 0.5×10⁶ cells per 35 mm dish, and cultured for two days. The cells were incubated in fresh DMEM medium supplemented with the noted concentrations of extract of 5 μg/ml, 10 μg/ml, or 20 μg/ml for 24 h at 37° C.

Cells were rinsed with ice-cold PBS and fixed with 4% paraformaldehyde for 10 min at room temperature, followed by permeabilization with 0.3% Triton x-100 for 10 min. After being washed with PBS three times, cells were incubated for 1 h in PBS containing 10% normal goat serum blocking solution. The cells were subjected to immunofluorescence staining with the specific antibodies (human myoblast cells studied for myostatin; rat L6 myoblast cells analyzed for mTOR) overnight at 4° C. The cells were then washed with cold PBS three times for 3 min each, and incubated with Alexa-labeled secondary antibodies (Invitrogen) at room temperature for 1 h. The cells were examined by fluorescence microscopy (a Nikon TE2000-S microscope, Nikon, Tokyo, Japan). For cell counts, five to ten random fields with approximately similar density of cells in each field were selected for analysis in each plate. Fluorescence intensities (with pixel values exceeding five times the standard deviation of the background) from these images were semi-quantitatively analyzed by densitometry (ImageJ software, NIH Image).

The Type-A polymer composition produced the significantly inhibited expression of myostatin relative to control at all concentrations tested, and mTOR protein levels were significantly higher in treated cells using both 10 μg/ml and 20 μg/ml concentrations (5 μg/ml was not tested). Thus, the data suggest that doubly linked procyanidin type-A polymers significantly enhance expression of mTOR and decrease expression of myostatin in skeletal muscle cells.

It will be appreciated by persons skilled in the art that the present invention is not limited to what has been particularly shown and described herein above.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified.

Patents and publications mentioned in the specification are indicative of the levels of those skilled in the art to which the invention pertains. These patents and publications are incorporated herein by reference to the same extent as if each individual application or publication was specifically and individually incorporated herein by reference for the entirety of their teaching.

The foregoing description is illustrative of particular aspects of the invention, but is not meant to be a limitation upon the practice thereof.

1. A process of increasing expression of a MTOR gene or a mTOR protein or decreasing expression of a MSTN gene or a myostatin protein in a subject comprising:
administering to the subject a composition comprising at least 0.5% Type-A polymers by weight, said Type-A polymers comprising A-Type doubly linked procyanidin oligomers of the catechins and/or epicatechins; and
increasing expression of a MTOR gene or a mTOR protein or decreasing expression of a MSTN gene or a myostatin protein in said subject by said step of administering.

2. The process of claim 1, wherein said A-Type doubly linked procyanidin oligomers comprise A-type doubly linked procyanidin dimers of catechins and/or epicatechins.

3. The process of claim 1, wherein said A-Type doubly linked procyanidin oligomers comprise A-type doubly linked procyanidin trimers of catechins and/or epicatechins.

4. The process of claim 1, wherein said A-Type doubly linked procyanidin oligomers comprise A-type doubly linked procyanidin tetramers of catechins and/or epicatechins.

5. The process of claim 1, wherein said A-Type doubly linked procyanidin oligomers comprise a mixture of A-type doubly linked procyanidin oligomers of catechins and/or epicatechins.

6. The process of claim 1 wherein the composition is administered orally, intravenously, by intramuscular injection, by intraperitoneal injection, or transdermally.

7. The process of claim 1, wherein said composition further comprises one or more vitamins, antioxidants, mTOR enhancers, myostatin inhibitors, or combinations thereof.

8. The process of claim 7 wherein said vitamin is vitamin A, vitamin B, vitamin C, vitamin D, vitamin E, vitamin K, or combinations thereof.

9. The process of claim 7 wherein said antioxidant is alpha lipoic acid, acai, astazanthin, wolfberry, glutathione, super oxide dismutase, or combinations thereof.

10. The process of claim 7 wherein the mTOR enhancer is a branched-chain amino acid.

11. The process of claim 10, wherein the branched-chain amino acid is leucine, isoleucine valine, or combinations thereof.

12. The process of claim 7 wherein the myostatin inhibitor is leucine, β-hydroxy β-methylbutyric acid, creatine, or combinations thereof.

13. The process of claim 1 wherein said A-Type doubly linked procyanidin oligomer is present at about 1-30 milligrams.

14. The process of claim 1 wherein said A-Type doubly linked procyanidin oligomer is present at about 3-10 milligrams.

15. The process of claim 1 wherein said composition is administered daily for a period of six weeks or more.

16. The process of claim 1 wherein said composition is administered one to three times daily.

17. The process of claim 1 further comprising quantifying the expression of a MTOR gene in said subject subsequent to said administering.

18. The process of claim 1 further comprising quantifying the expression of a MSTN gene in said subject subsequent to said administering.

19. The process of claim 1 further comprising quantifying the level of a mTOR protein expression in said subject subsequent to said administering.

20. The process of claim 1 further comprising quantifying the level of a myostatin protein expression in said subject subsequent to said administering.
